(19) United States
(12) Patent Application Publication
(10) Pub. No.: US 2011/0288252 A1
(43) Pub. Date: Nov. 24, 2011

(54) DIALKOXY- OR DIHYDROXYPHENYL RADICALS CONTAINING SILANES, ADHESIVES PRODUCED THEREFROM AND METHOD FOR PRODUCING SILANES AND ADHESIVES

(76) Inventors: Thomas Ballweg, Kreuzwertheim (DE); Somchith Nique, Eisingen (DE)

(21) Appl. No.: 13/129,478

(22) PCT Filed: Nov. 16, 2009
(86) PCT No.: PCT/EP09/65217

§ 371 Date: Aug. 2, 2011

(30) Foreign Application Priority Data
Nov. 17, 2008 (DE) 10 2008 057 684.0

(51) Int. Cl. C08F 30/08; C07K 7/06; C07F 7/18

(52) U.S. Cl. 526/238.1; 556/419; 556/420; 530/328; 526/279

(57) ABSTRACT

The present invention relates to silanes with the formula (I) RₐQ_{b}SiX_{4-a-b} (I) wherein the radicals and indices have the following meaning: R is optionally the same or different and identifies a straight-chain, branched-chain and/or cyclic alkyl, alkenyl, aryl, alkylaryl or arylalkyl group or a straight-chain or branched and/or cyclic organic radical with at least one organically polymerizable group, wherein the carbon chains can each be interrupted by one or more oxygen or sulfur atoms or carboxyl or carbonamide or amino groups or can hold one or more groups selected from carboxylic acid groups, carbonamide groups, amino groups, hydroxy groups and mercapto groups, at one of the ends of said carbon chains, Q is (C₆H₃)(OR¹)₂ or R³(C₆H₃)(OR¹)₂, where R¹ indicates hydrogen or a C₁C₄- alkyl group, and R³ is a substituted or unsubstituted carbon chain that is interrupted by either one or by multiple groups selected from —O—, —NH—, —NHC(O)—, —C(O)NH—, —C(O)NHC(O)—, —NHC(O)NH—, —C(O)O—, —NHC(O)O—, —C(O)—, —OC(O)NHC(O)O—, —S—, —S(O)—, —C(S)—, —C(O)S—, —C(S)NH—, —NHC(S)NH—, and/or is bonded to the radical (C₆H₃)(OR¹)₂ by way of one of said groups and/or comprises at least 7 carbon atoms in the chain. X is a group that can enter into a hydrolytic condensation reaction, forming Si—O—Si bridges, a is 0, 1 or 2, b is 1 or 2, and a+b together is 1, 2 or 3. Also, the invention relates to organically modified crystalline silica (hetereo)(partial) condensates that can be produced from silanes of formula (I), among other things, and that can be cured into an organic polymer in the presence of organically polymerizable groups. The crystalline silica (hetereo)(partial) condensates and polymers of the invention are suitable as adhesives, in particular for wet applications.

This invention refers to hydrolyzable and condensable silanes as well as resin-like poly-condensates or partial condensates (“organically modified silicic acid (hetero) poly (partial) condensates”) made there from. The silanes have at least one phenyl group (especially a 3,4-hydroxyphenyl group) substituted with a minimum of two hydroxyl or alkoxy groups, the polycondensates and partial condensates having as a rule numerous of these groups, often one such group for every silicon atom. The silanes and the organically modified silicic acid poly (partial) condensates can additionally contain organically polymerizable groups. The partially or fully hydrolyzable/condensed homo or hetero polycondensates of this invention that can be furthermore organically cross-linked if need be, are suited as adhesives for humid conditions, i.e. as materials that in the presence of water develop an adhesive effect towards many different substrates.

Especially in medicine, the dental field and in biotechnology, there is a great demand for suitable adhesives for fixing biological materials in place as well as for tissue (skin, bones) and individual cells in the presence of body fluids. Such a high-performance medical product is ideally characterized by: strong adhesion in the presence of water, high inner strength (cohesion), if possible mechanical properties adjustable to the surrounding tissue (bone, cartilage; muscle, skin), biocompatibility (i.e. minimal irritation potential and lowest possible cellular toxicity), a fast hardening mechanism, simple and efficient application and, where appropriate, the capability of being absorbed or detached once again.

To date, there are no adequate and extensive adhesives for humid conditions. The medical adhesives used are fibrin adhesives, albumin-based compounds, glutaraldehyde adhesives, cyanoacrilates, hydrogels and collagen-based compounds. From this series, the fibrin adhesives are the most widely used, but the risks of these blood-derived products are still regarded as significant so that their application field is limited to a few specialized surgical niche applications. Cyanoacrylates are even stronger adhesives than those made from fibrin and have been marketed as skin and wound adhesives for about 40 years (Histoacryl®), but their use is limited to external and short-term applications owing to their association with carcinogenicity, inflammation and infection potential.

As far back as in the 1970s, the central role of mussels was recognized for biomimetic, peptide-based, problem-solving approaches. Mussels are capable of adhering firmly and permanently to almost any substrate in the water under the most extreme conditions. They adhere not just to iron, wood and stone, but also to glass panels, paint surfaces or Teflon coatings. Even under the strongest salt-water surf, these crustaceans are able to remain attached for years to walls and piles with their sticky threads. This capability depends on the so-called sticky proteins, identified and nomenclated as Mefps (mussel adhesive foot proteins). Of all of them, Mefp-1 is the most intensively studied sticky protein with adhesive properties, comparable to synthetic cyanoacrylates and epoxy resins. It consists of 897 amino acids and is therefore a large protein. Inside this protein, an identical sequence of 10 amino acids turns up repeatedly, so that this “decapeptide” has been identified as the sub-unit mainly responsible for the adhesive effect. Within this decapeptide, in turn, the amino acid 3,4-dihydrophenylalanine (DOPA) plays a key role in the adhesive effect.

U.S. Pat. No. 4,745,169 describes silanes and siloxanes with dihydrophenyl radicals that are bound to the silicon through a substituted C1-C4 alkylene group, if applicable. The compounds are suggested for the manufacturing of light- and radiation-sensitive materials.

In order to make mussel proteins available as “bioadhesives”, the sticky proteins were extracted from the mussels employing protein extraction techniques and marketed as natural proteins (Sigma-Aldrich: “Adhesive Protein”; Swedish BioScience Laboratory: “MAP”; BD Biosciences Contech: “Cell-TAKM”). However, extraction is extremely time-consuming, as approx. 10,000 mussels are needed for extracting 1 g of the sticky protein—and this is not even the pure Mefp-1, but a mixture of the various mussel sticky proteins.

To circumvent the supply bottleneck, alternative extraction processes have been investigated. A described option: Recombinant protein techniques with which synthetic gene constructs find their application in bioreactor cultures, similar to insulin production (Genex Corporation's “Adhera Cell”). However, even with these techniques, only formulations with protein combinations from the mussel sticky proteins could be produced, associated with the corresponding limitations regarding purity, composition definition and therefore also the biological compatibility of the highly complex peptide structures. All these limitations have restricted the application of the sticky proteins to biotechnology's dissection fields.

A totally different alternative is the synthetic approach solution via the so-called solid phase synthesis, in which peptide sequences are built up through the successive stringing together of the respective amino acids from the elementary structural elements. This highly sophisticated combinational process is restricted to shorter peptides and consequently unsuitable for producing the sticky protein Mefp-1, which consists of 897 amino acids. The synthesis of the 10-component sub-unit (decapeptide), on the other hand, is possible and was already patented in 1986 (U.S. Pat. No. 4,585,585) and described in the literature (see Swerdloff, M. D. et al., Solid phase synthesis of bioadhesive analogue peptides with trifluoromethansulfonic acid cleavage from PAM resin, Int. J. Pept. Res. Vol. 33 (1989) 318-327. An enzymatic process for the production of proteins that contain DOPA from tyrosine-containing precursors is known (EP 242656 A2). Bioadhesive polyphenolic proteins have furthermore been described in the following applications: U.S. Pat. No. 5,015,677, WO 03/051418 A1, U.S. Pat. No. 5,410,023 and WO 2007/065742. The synthesis process, in particular, is still dominated nowadays by the Fraunhofer Institute for Manufacturing Technology and Advanced Materials (IFAM), which has expanded the method to an efficient process (see press release of the Fraunhofer IFAM, Bremen 2007, “Medizintechnik: Miesmuscheln liefern das Bioklebstoff der Zukunft [Medical Technology: Mussels Supply the Bioadhesive of the Future]”.

Striving to improve the efficiency and applicability of the sticky proteins, biomimetic and biomimetically-inspired approaches were pursued for anchoring the sticky proteins to matrices so synthetic bioadhesive could thus be generated. This is especially necessary when13 compared to the natural proteins—significantly shorter sub-units (such as the decapeptide or DOPA) should bring out their adhesive potential. It is known from the literature that it has already been possible to integrate them into polymers based on the water-soluble polyethylene glycols (PEGs) (see Lee, B. P., et al. “Synthesis and Gelation of DOPA-Modified Poly(ethylene glycol) Hydrogels. DOPA-modified hydrogels on the basis of PEG-diacrylate systems are a first known approach that goes beyond this, consisting in combining peptide-based adhesion mechanisms with a fast, light-induced hardening mechanism (see Lee, B. P. et al., Journal of Biomaterial Science—Polymer Edition, 15 (2004), 449-464.

The grafting of functional peptide sequences presupposes compatibility with the matrix and requires their sufficient hydrophilia. Most standard polymers are therefore unsuitable. Water-soluble polymers such as PEG lack internal cohesion. The introduction of an additive hardening mechanism leads to hydrogels of insufficient mechanical stability at best.

The task of this invention is to supply a resin system that has both the adhesive and cohesive properties of an adhesive effective in humid conditions and the starting materials for it. An advantage of the resin system should be its solvent-free production. Likewise advantageous should be the availability of the groups that make the adhesive effect possible only until the moment when the resin should unfold its adhesive effect.

The task is solved by supplying silanes with the formula (I)

RₐQ_{b}SiX_{4-a-b} (I)

wherein the radicals and indices mean the following:

R is, if applicable, the same or different and indicates a straight-chain, branched-chain and/or cyclic alkyl, alkenyl, aryl, alkylaryl or arylalkyl group with preferably 1 to 20 carbon atoms or, less preferred, a straight-chain or branched organic radical with at least one polymerizable group; preferably, this radical contains at least one C═C-double bond or a group accessible to a Michael condensation reaction with (more preferably) 2 to 25 carbon atoms; also in those cases in which R does not have this group, the carbon chain of R can be interrupted in specific arrangements by one or several oxygen or sulfur atoms or carboxyl or carbon amide or amino groups and/or carry at one of its ends one or several groups selected from among carboxylic acid groups, carbon amide groups, amino groups, hydroxyl groups and mercapto groups.

Q is the group —(C₆H₃)(OR¹)₂ or -R³(C₆H₃)(OR¹)₂, wherein R¹ stands for hydrogen or a C₁-C₄ alkyl group and R³ is a substituted or non-substituted carbon chain that is either interrupted by one or several groups selected from among —O—, —NH—, —NHC(O)—, —C(O)NH—, —C(O)NHC(O)—, —NHC(O)NH—, —C(O)O—, —NHC(O)O—, —C(O)—, —OC(O)NHC(O)O—, —S—, —C(S)—, —C(O)S—, —C(S)NH—, —NHC(S)NH— and/or bonded groups to the radical (C₆H₃)(OR¹)₂ through one of these groups and/or has at least 7, preferably at least 10, carbon atoms in the chain. In particular, R³ can contain within the chain or as substituent a radical B that has at least an organically polymerizable group that can especially undergo a polyaddition reaction.

X is a group that can enter into a hydrolytic condensation reaction by forming Si—O—Si bridges,

a is 0, 1 or 2

b is 1 or 2, and

a+b are together 1, 2 or 3 in a preferred embodiment 1.

The group (C₆H₃)(OR¹)₂ of the radical Q is responsible for the adhesive effect of the resin produced from these silanes: When R¹ is an alkyl radical, this adhesive effect is masked; when R¹ is a hydrogen, it is activated. The radical (C₆H₃)(OR¹)₂ is bound to the silicon atom either directly or through the spacer (-R³-), i.e. Q=(-R³)ₙ-(C₆H₃)(OR¹)₂ with n=0 or 1. The substituents (OR¹) are preferably bound to the phenyl group in ortho position with respect to each other, especially preferably if they are in p or m position with respect to the spacer. The conditions for the adhesive effect are especially good when the hydroxyl groups are in ortho position with respect to each other.

Within the meaning of the preceding definition, the spacer for R³ can be freely selected. When the carbon atom chain is interrupted by a coupling group (e.g. by —O—, —NH—, —NHC(O)—, —C(O)NHC(O)—, —C(O)—, —NHC(O)O— and similar ones, the adhesively active component is bound to the silicon with an isocyanate through esterifications or amidations or through the conversion of an acid group. In this way, cyclic carboxylic acid anhydride silanes of any ring size can undergo conversion with (HA) . . . (C₆H₃)(OR¹)₂ compounds, wherein HA is a hydroxyl, mercapto or amino group, in which case products are obtained in which the (C₆H₃)(OR)₂ group is bound to the silicon through an ester, thioester or amide group. If, on the other hand, isocyanate silanes are converted with (HO) . . . (C₆H₃)(OR¹)₂, products are obtained that are bound to the silicon through a urethane group. Through alternative, known conversions, it is possible to get to silanes with other coupling groups. Finally, the spacer can contain one or several groups B with organically cross-linkable groups which are either integrated to the spacer chain (divalent) or formed as side chain (monovalent). In this, the groups B consist of this group/these groups or they have them, in which case they are bound through a carbon atom chain and/or a coupling group such as an ester or amide group. The remaining constituents of B can be freely chosen. Examples for B are acrylate or methacrylate radicals.

The spacer can be either straight-chain or branched, have any chosen length, and/or have cyclen, in which case the reactive groups are located in the branches or can be bound to them. If its carbon chain is neither interrupted by one of the previously mentioned groups or bound to the (C₆H₃)(OR¹)₂ radical, it must have a minimum length of 7 carbon atoms. The number of carbon atoms above this quantity is not limited and the chain can have up to 50 C atoms, for instance. Longer spacers can contain polyethylene glycol units, for example, or have them as part of its structure. Reactive groups can occur, for instance, when the (HA) . . . (C₆H₃)(OR¹)₂ compound is an amino acid or peptide. When, for example, the reaction is conducted in such a way that the amino group reacts with a corresponding group bound to the silane—e.g. with an (activated) acid group or an anhydride—(at least) one free carboxylic acid group is preserved, which in turn can undergo further conversion.

If the silane of formula (I) contains two radicals Q, these can be the same or different.

In especially preferred invention embodiments, dopamine (1-amino-2-(3,4-dihydroxy)phenyl-ethane or DOPA (3,4-dihydroxy-phenylalanine) or the masked, alkoxylated form of these compounds can be bound directly or—preferably—via the spacer R³ to the silicon atom of a silane having the formula (I). The binding takes place preferably through the dopamine's amino group or the alpha-amino group of the DOPA. This canwhere appropriate, react with an activated acid group bound to the silicon atom via an alkylene group by forming an acid amide group, for example. Alternatively, the binding of DOPA (or another radical Q that carries a free amino group) can naturally take place through the carboxylic acid group, for example, via binding to an alkylene amino group bound to the silicon.

In another, especially preferred embodiment of the invention, a peptide containing a—if need be—masked dihydroxyphenyl group (substituted preferably in m and p position) or DOPA, preferably the decapeptide described by Swerdloff in 1989, bound to the silicon. Basically, the binding can take place through acid, hydroxyl or amino groups, as described above.

The reactive groups of the amino acid(s)/peptides can be protected and the peptides can, if needed, carry a urethane group in the N terminal and/or have one or several ethylene oxide groups as spacers, as shown in the Swerdloff peptide shown in a protected way:

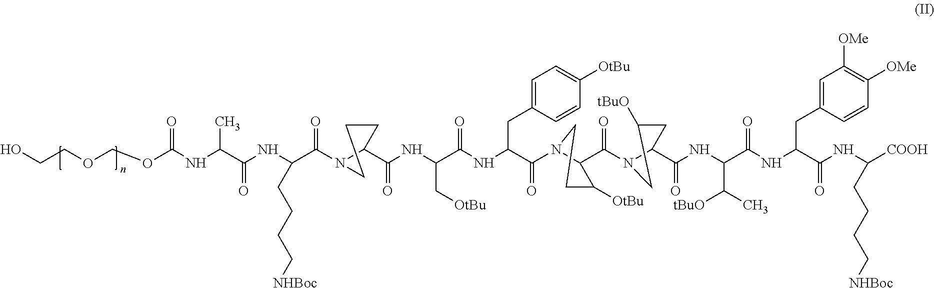

Optionally available amino groups can also—as shown in formula (II)—be protected according to classical protective group technique with the help of Boc groups. All these groups can be “deprotected” in subsequent reaction steps with the help of trifluoroacetic acid/boron tribromide, for example, thus obtaining a sticky product.

The X groups in the silanes having the formula (I) receive the name of inorganic network builders, as a silicic acid polycondensate network can be formed with the help of a subsequent hydrolytic condensation reaction. The specialist also knows what X can stand for. Apart from alkoxy, X can be, in case of need, a halide such as Cl, hydrogen, hydroxyl, acyloxy with preferably 2 to 5 carbon atoms, alkylcarbonyl with preferably 2 to 6 carbon atoms or an alkoxycarbonyl with preferably 2 to 6 carbon atoms. In some cases, X can also be NR″, where R means hydrogen, alkyl with preferably 1-4 carbon atoms or aryl with preferably 6-12 carbon atoms. Preferably, X is Cl or—better—a C₁-C₁₀ alkoxy group, especially preferred a C₁-C₄ alkoxy group and very much preferred methoxy or ethoxy.

In the silanes of formula (I), a is preferably 0 or 1; b is preferably 1. For this reason, the silanes of formula (I) especially preferably have the formulas (Ia) or (Ib)

QSiX₃ (Ia),

RQSiX₂ (Ib)

wherein R and X have the meaning given for formula (I) and X is preferably a C₁-C₄ alkoxy and especially methoxy or ethoxy.

The silanes of this invention can be hydrolytically condensed. As a rule, this reaction takes place under acidic or alkaline catalysis according to the known sol-gel process, in which inorganic-organic hybrid polymers are produced that are also named organically modified silicic acid (partial) condensates (ORMOCER®s), i.e. materials that combine inorganically cross-linkable or cross-linked with organically active structural units.

The hybrid polymers or poly (partial) condensates of the invention can be exclusively built up of from silanes having the formulas (I), (Ia) or (Ib); instead, they can be built up from further, mostly known organically cross-linkable silanes, for example, of from metallic compounds that can also be hydrolytically condensable and whose metal atoms can be incorporated into the polycondensate network. These polymers are called organically modified silicic acid heteropolycondensates. The poly (partial) condensates according to the invention can generally be called resins because they are either self-flowing or can be dissolved or dispersed in a suitable solvent (frequently water) or in an alcohol and harden after application on a substrate. The hardening can take place through drying or removal of the solvents or dispersants, through the cross-linking of existing organically cross-linkable groups and/or through a stronger (hydrolytic) condensation of such materials that at the moment of application are not fully hydrolyzed/condensed and thereby can also be named pre- or partial condensates.

Through the incorporation of structures that exert an adhesive effect under humid conditions into the matrix of such hybrid polymers, novel adhesives that harden under humid conditions can be produced. By anchoring into a adjustable matrix with regard to the mechanical and wetting properties, and by combining biological adhesion with polymer-chemical hardening mechanisms (chemically- or light-/UV-induced), a controlled and efficient application method is made possible that overcomes the disadvantages of the methods that use peptide-based adhesive employed so far.

The previously described resins or organically modified silicic acid (partial) condensates can also be obtained in another way; specifically, through the conversion of already pre-condensed silanes with the corresponding compounds that contain the (C₆H₃)(OR¹)₂ group described in detail above. The reaction pathways follow those previously described for silane production; basically, the binding steps of the sticky component to the silyl unit and the hydrolytic silane condensation are in this case exchanged.

As far as the matrix of the organically modified silicic acid (hetero) polycondensates should be accessible to an organic polymerization in the open or protected sticky component (i.e. to a polymer chemistry hardening mechanism as explained above), the groups needed for this can be brought into the system in various ways:

In a first variant, these groups are bound to the silanes of formula (I) or to a portion thereof, which means that they are located in the same silicon atoms that also carry the Q group. This can be achieved both ways: either by using conversion products of the sticky component with silanes of formula (I) in which R is a straight-chain or branched radical with at least one organically polymerizable group or with corresponding (pre-) condensates of these silanes. However, it is instead preferable to use (or, if need be, also additionally) a silane with the formula (I) (or a (pre-) condensate thereof) in which the radical Q contains at least one monovalent or divalent group B with at least one organically polymerizable group, as described above.

Alternatively (if need be, also additionally), the silanes of formula (I) can be co-condensed together with other, second silanes that carry one or several (preferably two, but if need be, more) organically polymerizable R′ groups, many of which are known in the state of the art. Advantageously, the organic R, R′ radicals/groups or B with polymerizable groups are those with at least one reactive ring or at least one reactive double bond; under the influence of initiators, heat and/or actinic radiation, they cause a radical, anionic or cationic polymerization (“addition polymerization” in English). Nonetheless, the polymerizable group may also undergo another polyreaction such as a condensation reaction (forming an ester or amide, for example) or something similar. Advantageously, R, R′ and B have at least an epoxy group and/or at least a C═C double bond (and thereby 2 to preferably 50, even better up to 25 carbon atoms) that can be part of a vinyl, allyl, norbornene, acryl and/or methacryl group, for example. In favorable instances, every double bond is part of a Michael system, very preferable a part of an acrylate or methacrylate group, acrylamide or methacrylamide group. In another preferred embodiment, two or even three Michael systems can exist, bound to a radical or distributed among several radicals per silane molecule. In these radicals, the polymerizable groups can be directly bound to the silicon through carbon atoms; however, the connecting carbon chain can also be interrupted by heteroatoms or groups such as —O—, —S—, —S(O)—, —NH—, —NHC(O)—, —C(O)NHC(O)—, —C(O)O—, —NHC(O)O— or similar ones. Its carbon skeleton can be exclusively aliphatic, specifically with open and/or closed structures, but also have one or several aromatic core(s) or condensed systems or triazine groups or similar ones (e.g. bisphenol A structures or the like). Furthermore, the groups can be freely substituted with acid, acid amide, ester, urethane, or amino groups, for example.

As mentioned above, group B can be monovalent or divalent. In the first case, it is a side group of the spacer; in the second case, the group is integrated into the Q spacer.

For preparing the condensates, the second silanes can be partially or fully hydrolyzed together with or separated from the silanes with the formula (I). The condensation that follows the hydrolysis can likewise be partial or total.

Accordingly, the invention supplies an organically modified silicic acid (hetero) (partial) condensate with sticky components containing structural units of formula (II)

RₐQ_{b}Si(OR²)_{4-a-b} (II)

wherein the radicals R and Q and the indices a and b are defined for the formula (I) as above and the radicals R² are the same or different and at least sand for a bond to another silicon atom and moreover represent a hydrogen atom, an alkyl group with 1 to 10 carbon atoms or a bond to another metal atom that can be incorporated into silicic acid heteropolycondensates.

The silanes with the formula (I) can contain any radicals R and X for achieving the suitable properties of the organically modified silicic acid (hetero) (partial) condensates. In the literature of inorganic-organic materials containing silicon atoms (e.g. those already being sold in the market under the name “ORMOCERE®”), a lot has been written about the respective properties that the respective silane radicals confer to the condensate or organically polymerized network, so that no detailed explanations are necessary here. As mentioned above, the X groups very generally designate the hydrolyzable radicals. With these groups, which are also known as inorganic network formers, physical properties of the forming network such as stability, hardness and flexibility are set in combination with possibly available organic network formers (in those cases, in which Q or R have at least one organically polymerizable group). Non-organic polymerizable groups R are known as network modifiers; with their selection, a series of properties can also be influenced.

Further variations are obtained by incorporating additional metal atoms such as boron, aluminum, titanium or germanium that can be added in the form of their hydrolytic alkoxy compounds to the compounds to be hydrolyzed.

When the silanes used for this purpose contain organically polymerizable groups, the silicic acid (hetero) (partial) condensate according to the invention can then be organically cross-linked—depending on the groups used, for example by irradiation with actinic radiation, using redox catalysts or heat, as known in the state of the art. As a result of that, a second, organically bridged network is formed that interpenetrates or superimposes the first. The polymers obtained in this way are characterized by further improved mechanical stabilities.

In rare cases, it is also possible to make the silanes of formula (I) according to the invention undergo an organic polymerization as long as they have an organically polymerizable group, if need be in the presence of additional, organically polymerizable silanes and to add the polymer obtained in this way only after a hydrolytic condensation and, if necessary, a deprotection of the masked groups.

When applying the silanes according to the invention, it is therefore possible to make use of one or two different cross-linking mechanisms: Whereas adhesion can be triggered through the dihydroxyphenyl groups in the case of surface contact, through complexation, via oxidants or enzymatically, a cohesive matrix hardening can be started with light, UV or redox. Afterwards, a final subsequent cross-linking can take place through diffusing complexation reagents (e.g. Fe(III) ions). In the course of this, the dihydrophenyl groups of the adhesive compound far from the surface, not involved in the adhesion and therefore still free or not reacted, are complexed by multivalent cations triggered from the surface (e.g., bone, tooth) so that they, for their part, are (also) able to additionally contribute to the cohesive stability of the bonding. (This subsequent cross-linking phase can take a long time.) Through the amphiphile build up of the silicic acid (partial) condensates modified with the sticky component such as dopamine, DOPA or a corresponding peptide, orientation effects also occur at the interfacial surfaces that can greatly increase the adhesive effect. In this way, an adhesive is obtained that combines the hardening mechanisms known from bonding technology with the capability of adhering in humid conditions.

The inorganic-organic hybrid polymers or organically modified silicic acid poly (partial) condensates of this invention enrich the ORMOCER® class of materials in which inorganically cross-linkable structures are combined with organically cross-linkable ones that therefore take an intermediate position between classical polymers, silicones and glasses. Their dual character allows a property tuning that makes them an adaptable work material offering a wide variety of properties and processing options. This intermediate position predestines them for meeting complex requirement profiles in the boundary field of organic and inorganic, respectively water-based chemistry, which in the past could be documented in successful product developments in the field of light-hardening dental filling composites or scratch-resistant coatings. This also promises many different areas of application for the materials. The combination of peptide chemistry and sol-gel chemistry of the ORMOCER®s opens up, among other things and owing to the amphiphilic properties of the silanes having the formula (I) and the (pre-) condensates that can be produced from them, the possibility of variably setting the polar character of these condensates and to supply solvent-free, dissolved or dispersed resins that, if need be, can be timely “sharpened” prior to their use (by removing the protecting groups from the sticky components) and use them as adhesives on a moist undersurface or in a humid environment and/or for other purposes such as improving substrate biocompatibility. It has been shown that organically modified silicic acid polycondensates also based on silanes can be thoroughly condensed with three inorganically condensable X groups under suitable reaction control and nonetheless can have a highly viscous, plastic consistency that can flow in a fully or almost solvent-free state without being gelled. This phenomenon can best be explained with the non-random crossing theory, i.e. the formation of ordered network structures as known in the context of the silsesquioxanes.

The most important advantage of this invention is that a skeletal structure is offered to the groups, amino acids or peptides that allows bonding and imparts cohesion and fullness to the adhesive that potentiates the adhesive effect of the groups that cause the bonding, thus making an adhesive that can be applied with reasonable effort available for use in medical, biotechnological and other technical tasks as well. The inner stability and cohesion of the adhesive that cannot be achieved with the approaches used so far is given. if need be, by an additive, e.g. light- or UV-induced hardening mechanism as described above that also allows easy and quick application.

The adhesive according to the invention can be used in many fields for attaching materials in dry and humid conditions, especially in medical technology. Tissue adhesives made from these materials can replace post-surgical sutures or fixation aids for bone ligaments, bone tendons and similar uses. Biocompatible bone adhesives for load-supporting areas have a high application potential. Further fields of use are dentistry (bonding agents), ophthalmology (retinal repairs) and the biotechnology for enzyme immobilization without loss of enzyme activity, in situ hybridizations or immunoassays. The adhesive according to the invention is also particularly useful for attaching non-biological materials, for example in the electronics, electrical engineering and optics fields, where small structural parts with high adhesive power are brought together.

The invention will now be explained in more detail with the help of examples.

### Example 1

This example is about the production of a resin based on a DOPA-modified organosilane suitable for co-condensation with an ORMOCER® that can be light-/UV-hardened (hydrolytically condensed silanes). Within the decapaptide, DOPA is the most active and most thoroughly researched amino acid with regard to adhesive effect.

Synthesis

25.35 mmol of 3,4-dihydroxy-L-phenylalanine and 25.35 mmol of 3-(triethoxysilyl)propyl succinic acid anhydride were suspended in 15 mL anhydrous dimethyl sulfoxide. The suspension was heated up under nitrogen at 80° C. for obtaining a homogenous solution after a short time. After a 3-hour agitation time, conversion has been completed. Once the solution has cooled off, it was diluted with 25.35 mL ethanol and stirred with aqueous ammonium fluoride solution until full hydrolysis had taken place. Afterwards, most of the ethanol was removed in the rotary evaporator. From the concentrated solution obtained, the product was precipitated with methylene chloride, washed with water and vacuum dried.

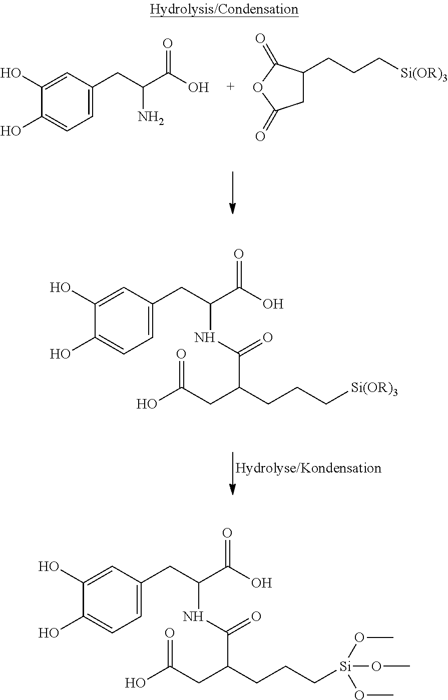

In this example, the binding of the sticky components or of the silane that supplies the adhesive effect takes place exclusively inorganically through co-condensation in the sol-gel process; there are no groups available that could be accessible to a polyaddition. The example shows a very simple form of the amino acid functionalization of an organosilane that on the one hand does not have organic reactive units and on the other hand does not need protective group techniques because of the simplicity of the individual amino acid. It shows not only the production of a DOPA-modified organosilane precursor, but is also an example for producing solvent-free resins based on this precursor through ordered network formation in the controlled sol-gel process.

### Example 2

This is an example for carrying out a co-condensation of the DOPA-modified organosilane described in Example 1 with a silane that carries organic, cross-linkable methacrylate groups in the sol-gel process. The result is a resin that can be hardened with light /UV in which DOPA-modified organosilanes are inorganically bound in a cross-linked way.

Mixture A

12.5 mmol of 3-isocyanatepropyltriethoxysilane are added drop-wise in such a way to a mixture of 12.5 mmol glycerin-1,3-dimethacrylate and 0.06 mmol dibutyltin dilaurate under nitrogen, cooling in the ice bath under light exclusion and agitation, that the temperature of 15° C. is not exceeded. After ending the addition, the mixture is stirred at 30° C. After 18 hours of stirring time, the conversion has been completed.

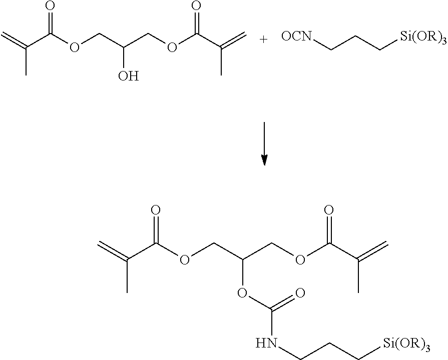

Mixture B

25 mmol of 3,4-dihydroxy-L-phenylalanine and 25 mmol of 3-(triethoxysilyl)propyl succinic acid anhydride were suspended in 15 mL anhydrous dimethyl sulfoxide. The suspension was heated up under nitrogen at 80° C. for obtaining a homogenous solution after a short time. After a 3-hour agitation time, conversion has been completed.

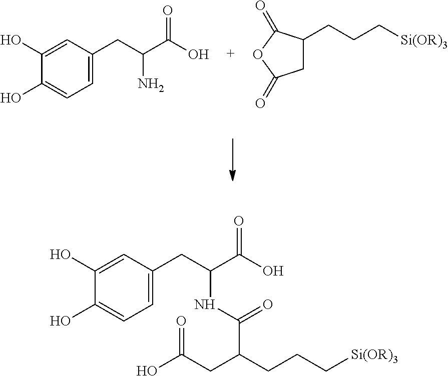

Components A and B were dissolved in 37.5 mL ethanol and stirred with aqueous ammonium fluoride solution until full hydrolysis occurred. Afterwards, most of the ethanol was removed in the rotary evaporator. From the concentrated solution obtained, the product was precipitated with water, washed with water and dried in a high vacuum.

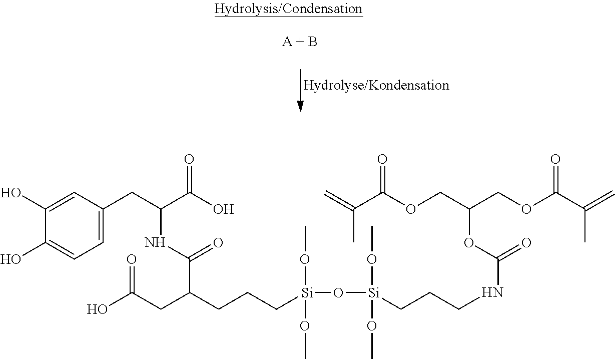

The product of this example is a condensate accessible to another purely organic cross-linking that can be caused in the conventional way (e.g. with initiators and irradiation). The advantage lies in the fact that, if necessary, the already pre-condensed resin can be applied on a surface to be glued and afterwards hardened through irradiation.

### Example 3

This is an example of a decapeptide-modified organosilane, where the decapeptide undergoes conversion with a silanisocyanate through an OH group in a polyethylene glycol spacer, the product being a silane of formula (I). The integration to a silicic acid polycondensate matrix can take place through co-condensation with the excess methacrylate silanes available, as shown in Example 2.

Synthesis

0.1 mmol of fully protected decapeptide and 0.6 mg of dibutyltin laurate were dissolved in 2 mL anhydrous acetobitrile. Under nitrogen and agitation, 0.1 mmol isocyanatepropyltriethoxysilane is added drop-wise. The reaction mixture was stirred for 20 hours at 30° C. and the solvent evaporated under a vacuum. The remaining product was dissolved in acetic acid ethyl ester and hydrolyzed with aqueous ammonium fluoride solution. After removing the solvent, the peptide is dried under a vacuum.

Formula of the protected decapeptide:

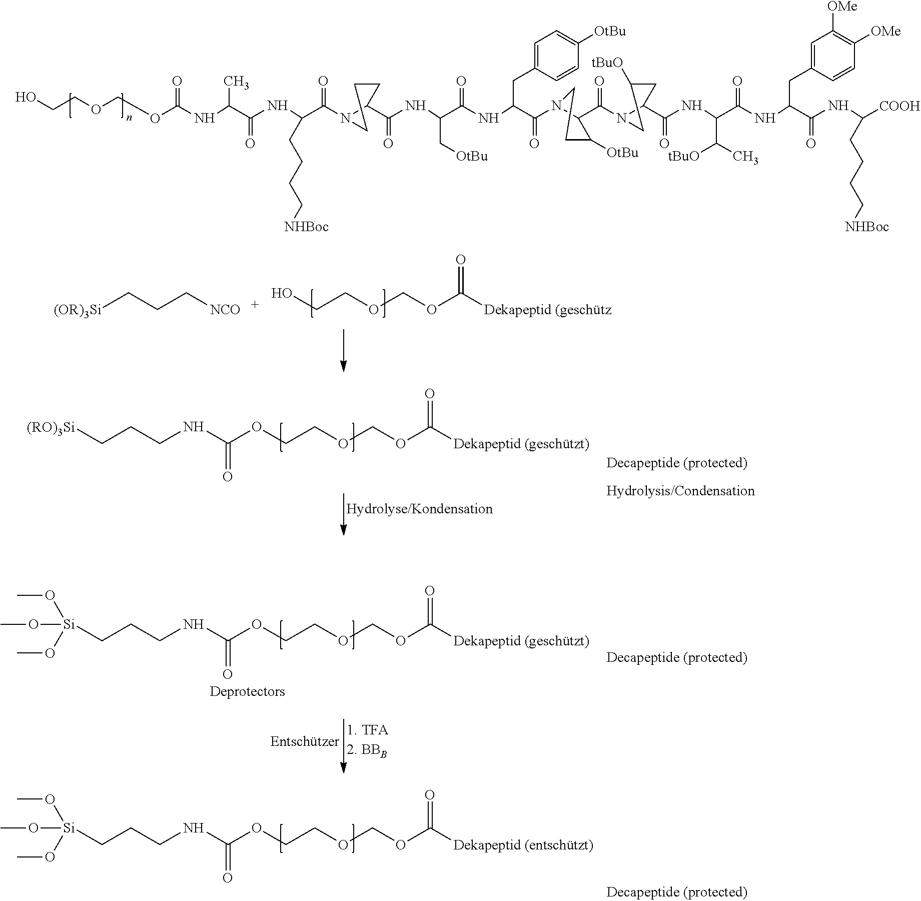

For splitting off the amino protective groups, the peptide was treated with a solution that consisted of 3.6 mL trifluoroacetic acid and 0.2 mL water.

The splitting off of the methoxy groups was done by treating the peptide with borotribromide: 0.01 mmol of the decapeptide was dissolved in 30 mL dry choroform and with the help of the water aspirator vacuum, the solution was alternately degassed and flooded with argon for approx. 5 min., then cooled to −25° C. under argon. Afterwards, 0.4 mmol of borotribromide (1M in methylene chloride) were added drop-wise in such a way that the temperature did not increase above −15° C. Thereafter, the solution was stirred under argon at room temperature but under light exclusion and stirred with methanol and water. The solvents were removed under vacuum. The aqueous peptide solution was frozen in liquid nitrogen and freeze dried.

The deprotected decapeptide only has the formula:

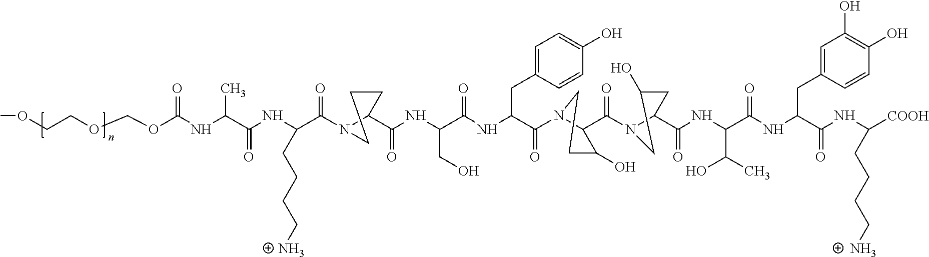

The protective groups present during binding and condensation enlarge the volume that the decapeptide occupies somewhat. The decapeptide could also be bound directly to the silane and incorporated into the matrix even without PEG spacer.

### Example 4

This example shows the production of a resin with structural units having the formula (II) through conversion of a previously (partially) condensed silicic acid polycondensate (ORMOCER®s) that contains polymerizable methacrylate groups and free acid groups with dopamine.

Dopamine as part of the amino acid DOPA is the smallest unit whose active adhesive effect is recorded in the materials according to the invention that fall within the framework of the examples. As far as the silicic acid polycondensate used as starting material is a pre-polymer or partial condensate with a molecular weight that isn't too large, it can be mixed with additional silane resins that can be hardened with light/UV when needed and then finally condensed. The organic hardening with light/UV can also take place in the same way after admixing such pre-condensed silane resins.

The product of the conversion shown below is a highly viscous, amber-colored resin. The underlying synthesis sequence has the advantage that the groups that make the adhesion possible do not have to be in a protected state during the conversion.

Synthesis

7 mmol of ORMOCER and 17.5 mmol of triethylamine were dissolved in 20 mL anhydrous methylene chloride. Under argon atmosphere at room temperature, 7 mmol of N,N′-dissuccinimidyl carbonate were added. The reaction mixture was stirred at room temperature for 2 hours, admixed with 7 mmol dopamine, and stirred for 24 hrs. After evaporating the solvent to a small bulk, the residue was absorbed in 20 mL methylene chloride and washed with water. Afterwards, the solvent was distilled off and the product vacuum dried.

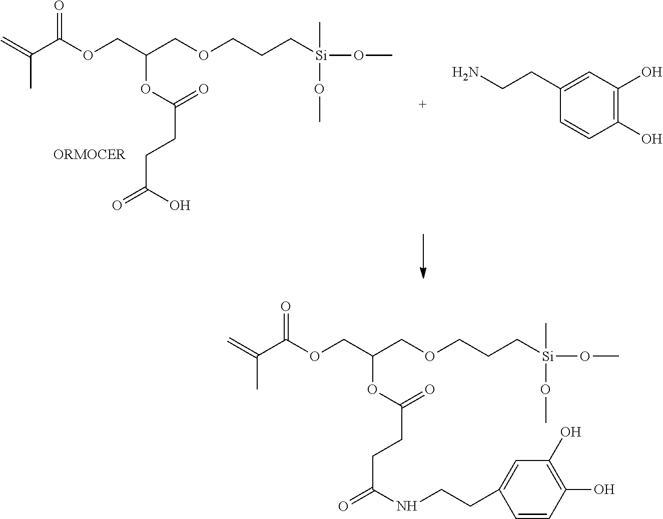

The condensate obtained in this way was tested for adhesiveness (stickiness) as follows: Based on a dimethylacrylate silane, it was mixed in proportion 1:4 in a silicic acid poly partial condensate (ORMOCER® resin) in which 1.5% of the photoinitiator Lucirin TPO had been dissolved; the constituents were thoroughly mixed and then 2 glass panels containing Fe were sanded, rinsed off with clear water, and dried. Optionally, the glass panels were steamed. One drop of resin was pressed between the panels. After waiting for 5-10 minutes (so the inorganic orientation and interaction could take place), the compound was hardened by irradiating it with a Hönle spotlight (2 min.). The testing of the adhesive attachment was done with a Zwick universal testing machine in the pressure mode.

The results are shown in Table 1. Whereas as a result of the polymerization shrinkage that accompanies the hardening process the unmodified ORMOCER resin peels off from the glass after the UV-induced hardening and therefore shows no measurable adhesion, the dopamine-modified resin (named “bioglue” in Table 1) adheres well compared to the commercial glues measured and prepared under the same conditions. The adhesive effect is still present in the case of the glass panels previously steamed with water vapor (indicated in Table 1 as “bioglue moist”).

TABLE 1

| Glue | Adhesive strength [MPa] |
|---|---|
| 2K epoxy resin | 5.1 ± 0.1 |
| Bioglue | 4.8 ± 0.5 |
| Bioglue moist | 5.2 ± 0.6 |
| Pattex | 0.80 ± 0.05 |
| Double adhesive tape | 0.19 ± 0.01 |

Without wanting to rely on the following theoretical considerations, the inventors suspect that the adhesive effect is based on an orientation of the relatively polar dopamine hanging on a moving chain with respect to the glass surface.

### Example 5

This example shows the production of a resin based on a decapeptide-modified methacrylate silane or DOPA-modified ORMOCER®. It is a variant of the decapeptide-modified organosilane according to Example 3. In this example, the decapapetide has no spacer, but is incidentally fully protected. The coupling takes place via the alpha-amino group of the N-terminal glycine and follows the dopamine coupling from Example 4 with a bifunctional organosilane precursor, which is equally suitable for co-condensation and co-polymerization with ORMOCER®s that can be light-/UV-hardened.

0.76 mmol ORMOCER and 1.9 mmol triethylamine were dissolved in 5 mL anhydrous methylene chloride. Under argon atmosphere at room temperature, 0.7 mmol of N,N′-dissuccinimidyl carbonate were added. The reaction mixture was stirred at room temperature for 2 hours, admixed with 0.7 mmol dopamine and stirred for 24 hrs. After evaporating the solvent to a small bulk, the residue was absorbed in 20 mL methylene chloride and washed with water. Afterwards, the solvent was distilled off and the product vacuum dried. For splitting off the amino protective groups, the peptide was treated with trifluoroacetic acid. The hydroxyl protective groups were split off using boron tribromide.

Formula of the protected decapeptide:

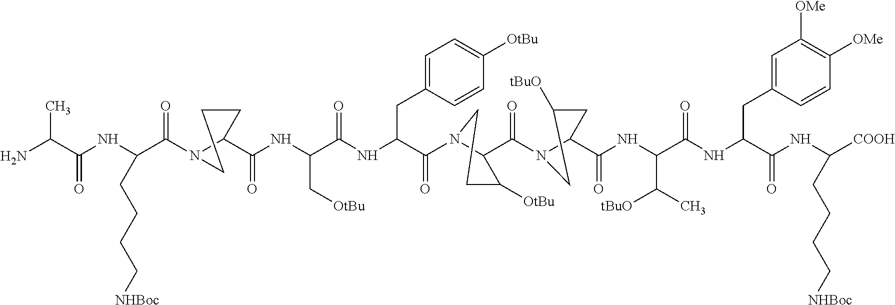

Formula of the deprotected decapeptide:

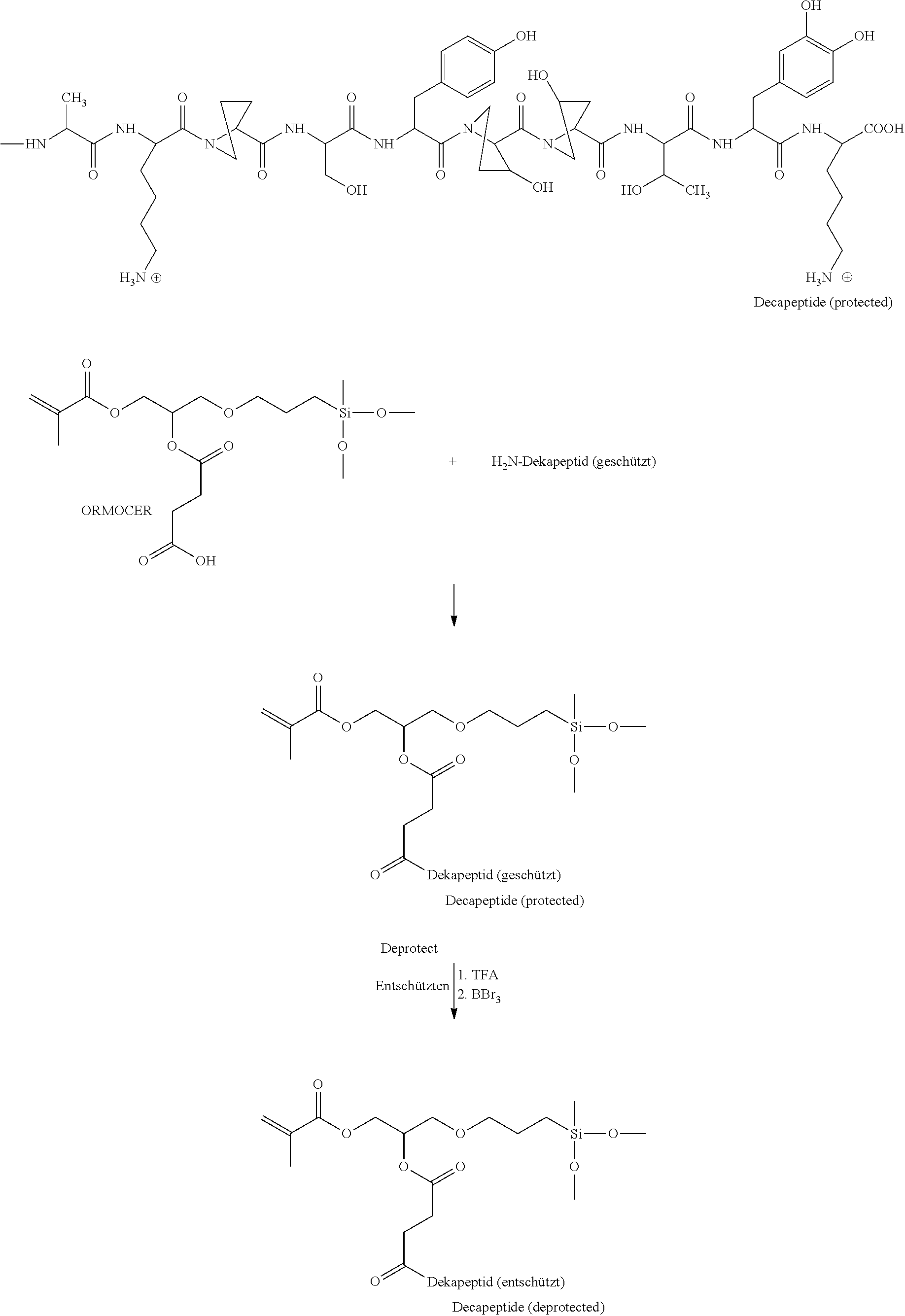

1. Silane with the formula (I)
RₐQ_{b}SiX_{4-a-b} (I)
wherein the radicals and indices mean the following:
R is, if applicable, the same or different and indicates a straight-chain, branched-chain and/or cyclic alkyl, alkenyl, aryl, alkylaryl or arylalkyl group or a straight-chain or branched and/or cyclic organic radical with at least one organic polymerizable group, in which case each carbon chain can be interrupted by one or several oxygen or sulfur atoms or carboxyl, carbon amide or amino groups or can carry one or several groups selected from among carboxylic acid groups, carbon amide groups, amino groups, hydroxide groups and mercapto groups on one of its ends,
Q is (C₆H₃)(OR¹)₂ or R³ (C₆H₃)(OR¹)₂, wherein R¹ stands for hydrogen or a C₁-C₄ alkyl group and R³ for a substituted or non-substituted carbon chain that is either interrupted by one or several groups selected from among —O—, —NH—, —NHC(O)—, —C(O)NH—, —C(O)NHC(O)—, —NHC(O)NH—, —C(O)O—, —NHC(O)O—, —C(O)—, —OC(O)NHC(O)O—, —S—, —S(O)—, —C(S)—, —C(O)S—, —C(S)NH—, —NHC(S)NH— and/or bonded through one of these groups with the radical (C₆H₃)(OR¹)₂ and/or has at least 7 carbon atoms in the chain.
X is a group that can enter into a hydrolytic condensation reaction by forming Si—O—Si bridges,
a is 0, 1 or 2
b is 1 or 2, and
a+b are together 1, 2 or 3.

2. Silane according to claim 1, wherein R³ is a carbon chain with 1 to 40 C atoms interrupted by one or several groups selected from among —O—, —NH—, —NHC(O)—, —C(O)NH—, —C(O)NHC(O)—, —C(O)O—, —NHC(O)O— and/or carries one or several additional substituents and/or polymerizable groups.

3. Silane according to claim 2, wherein Q contains a minimum of one monovalent or divalent group B with at least one organic polymerizable group, in which case the organic polymerizable group is selected from among groups having at least one C═C double bond or at least a group accessible to a Michael condensation reaction, preferably a (meth)acrylate group or a (meth)acrylamide group.

4. Silane according to claim 2 or 3, wherein R³ is a carbon chain interrupted by at least one of the groups —NHC(O)—, —C(O)NH—, —C(O)O— and/or one or several oxygen atoms, in which case the latter are present preferably as polyethylene oxide units.

5. Silane according to one of the claims 2 to 4, wherein Q contains the radical —C(O)—NH—CHR⁴—CH₂—(C₆H₃)(OR¹)₂, in which case R⁴ is hydrogen or COOH or COO⁻.

6. Silane according to one of the preceding claims with the formula (Ia) or (Ib)
QSiX₃ (Ia),
RQSiX₂ (Ib)
wherein X is a C₁-C₄ alkoxy and especially methoxy or ethoxy and R has the meaning given in claim 1 for formula (I).

7. Organically modified silicic acid (hetero) (partial) condensate containing structural units of formula (II),
RₐQ_{b}Si(OR²)_{4-a-b} (II)
wherein the radicals R and Q and the indices a and b are defined in the same way as for the formulas (I), (Ia) and (Ib) in claims 1 through 6 and the radicals R² are, if applicable, the same or different and mean at least partially a bond to another silicon atom and incidentally represent a hydrogen atom, an alkyl group with 1 to 10 carbon atoms or a bond to another metal atom that can be incorporated into silicic acid hetero polycondensates.

8. Organically modified silicic acid (hetero) (partial) condensate according to claim 7 containing groups accessible to an organic polymerization.

9. Organically modified silicic acid (hetero) (partial) condensate according to claim 8, wherein the groups accessible to an organic polymerization are selected from among those that have at least one reactive ring or at least one reactive double bond, and under the influence of initiators, heat and/or actinic radiation undergo a radical, anionic or cationic polymerization.

10. Organically modified silicic acid (hetero) (partial) condensate according to claim 8 or 9, wherein at least one part of the groups accessible to an organic polymerization are bound to silicon atoms that furthermore carry at least a Q radical and/or wherein at least one part of the groups accessible to an organic polymerization are parts of the Q radicals.

11. Organically modified silicic acid (hetero) (partial) condensate according to one of the claim 8 or 9, wherein at least one part of the groups accessible to an organic polymerization is bound to silicon atoms that do not carry the Q radical.

12. Organically modified silicic acid (hetero) (partial) condensate according to one of the claims 8 to 11, wherein at least one part of the groups accessible to an organic polymerization are bound to a silicon atom that can be interrupted by one or several heteroatoms and/or groups selected from among —O—, —S—, —S(O)—, —NH—, —NHC(O)—, —C(O)NHC(O)—, —C(O)O—, —C(O)NH—, —NHC(O)O—.

13. Organically modified silicic acid (hetero) (partial) condensate according to one of the claims 7 through 12, in which R¹ in the Q group is hydrogen.

14. Homo or heteropolymer comprising an organically modified silicic acid (hetero) (partial) condensate according to one of the claims 8 through 10, whose groups accessible to an organic polymerization are at least partially available in polymerized form.

15. Process for producing a silane with the formula (I) as indicated in claim 1, wherein Q stands for R³-(C₆H₃)(OR¹)₂ and R³ is a carbon chain with 1 to 40 C atoms interrupted by at least one group selected from among —C(O)NH—, —C(O)O— and —C(O)S— that encompasses the conversion of a compound with the formula (III)
W-R⁷-(C₆H₃)(OR¹)₂ (III)
wherein W stands for NH₂, OH or SH and R⁷ is any divalent organic radical, with a silane having the formula (IV),
RₐY_{b}SiX_{4-a-b} (IV)
wherein Y stands for R⁵-COA and R⁵ is an alkylene group with 1 to 10 carbon atoms and COA is a carboxylic acid group, an activated carboxylic acid group or a radical carrying a carboxylic acid anhydride group, where appropriate in the presence of an acid amide formation promoting agent.

16. Process for producing a silane with the formula (I) as indicated in claim 1, wherein Q stands for R³-(C₆H₃)(OR¹)₂ and R³ is a carbon chain with 1 to 40 C atoms interrupted by at least one —NHC(O) group encompassing the conversion of a compound with the formula (III)
W-R⁷-(C₆H₃)(OR¹)₂ (III)
wherein W stands for OH and R⁷ is any divalent organic residue with a silane of the formula (V)
RₐZ_{b}SiX_{4-a-b} (V)
wherein Z stands for R⁶-NCO and R⁶ is an alkylene group with 1 to 10 C atoms.

17. Process for producing an organically modified silicic acid (hetero) (partial) condensate according to claim 7 or 8, characterized in that a silane of the formula (I) is subject to at least a partially hydrolytic condensation reaction, if need be in the presence of more silane compounds.

18. Process according to claim 17, characterized in that the at least partially occurring hydrolytic condensation reaction takes place in the presence of a second silane compound that carries at least an organically polymerizable group.

19. Process for producing an organically modified silicic acid (hetero) (partial) condensate according to claim 7, wherein Q stands for R³-(C₆H₃)(OR¹)₂ and R³ is a carbon chain with 1 to 40 C atoms interrupted by at least one group selected from among —C(O)NH—, —C(O)O— and —C(O)S— characterized in that an organically modified silicic acid (hetero) (partial) condensate is provided with structural units of the formulas (VIb) wherein R, R², a and b have the meaning given in claims 1 and 7 for the formulas (I) and (II) and Y has the meaning given in claim 14 for the formula (IV),
with a compound having the formula (II)
W-R⁷-(C₆H₃)(OR¹)₂ (II)
wherein W stands for NH₂, OH or SH and R⁷ is any divalent organic radical.

20. Process for producing an organically modified silicic acid (hetero) (partial) condensate according to claim 7, wherein Q stands for R³-(C₆H₃)(OR¹)₂ and R³ is a carbon chain with 1 to 40 C atoms interrupted by at least one —NHC(O)— group, characterized in that an organically modified silicic acid (hetero) (partial) condensate is provided with structural units of the formulas (VIIb)
RₐZ_{b}Si(OR²)_{4-a-b} (VIIb)
wherein R, R², a and b have the meaning given in claims 1 and 7 for the formulas (I) and (II) and Z the meaning given in claim 16 for the formula (V) and this one, converted, is with a compound having the formula (III)
W-R⁷-(C₆H₃)(OR¹)₂ (III)
wherein W stands for OH and R⁷ is any divalent organic radical.

21. Process for producing a homopolymer or heteropolymers according to claim 14 encompassing the polymerization of existing groups accessible to an organic polymerization of an organically modified silicic acid (hetero) (partial) condensate according to one of the claims 8 through 12.
